(19) United States
(12) Patent Application Publication
(10) Pub. No.: US 2026/0098042 A1
(43) Pub. Date: Apr. 9, 2026

(54) PHARMACEUTICALLY ACCEPTABLE ACID SALT OF FREE BASE OF GLP1 RECEPTOR AGONIST, AND PREPARATION METHOD THEREFOR

(71) Applicant: Hangzhou Zhongmei Huadong Pharmaceutical Co., Ltd., Zhejiang (CN)

(72) Inventors: Haiwen HU, Zhejiang (CN); Li FANG, Zhejiang (CN); Fan HU, Zhejiang (CN); Xinjie ZHOU, Zhejiang (CN); Fenfen CHEN, Zhejiang (CN)

(21) Appl. No.: 19/304,270

(22) Filed: Aug. 19, 2025

Related U.S. Application Data
(63) Continuation of application No. 17/995,091, now Pat. No. 12,421,245.

(30) Foreign Application Priority Data
Apr. 1, 2020 (CN) 2020010248957.3

(51) Int. Cl. C07D 491/056

(52) U.S. Cl. CPC C07D 491/056; C07B 2200/13

(57) ABSTRACT

The invention provides pharmaceutically acceptable acid salts of (S)-2-(3S,8S)-3-(4-(3,4-dichlorobenzyloxy)phenyl-7-((S)-1-phenylpropyl)-2,3,6,7,8,9-hexahydro-[1,4]-dioxino[2,3-g]isoquinolin-8-ylformylamino)-3-(4-(2,3-dimethylpyridin-4-yl)phenyl) propionic acid (“OAD2”) and the preparation method thereof. The pharmaceutically acceptable acid salts of OAD2 provided herein may be useful in the treatment of various conditions and metabolic disorders including, but not limited to, type 2 diabetes.

### TECHNICAL FIELD

The invention belongs to the technical field of medicine, and particularly relates to pharmaceutically acceptable acid salts of (S)-2-(3S,8S)-3-(4-(3,4-dichlorobenzyloxy)phenyl-7-((S)-1-phenylpropyl)-2,3,6,7,8,9-hexahydro-[1,4]-dioxino[2,3-g]isoquinolin-8-ylformylamino)-3-(4-(2,3-dimethylpyridin-4-yl)phenyl)propionic acid (“OAD2”), and the preparation methods thereof. The pharmaceutically acceptable acid salts of the invention may be useful in the treatment of various conditions and metabolic disorders including, but not limited to, type 2 diabetes.

### BACKGROUND OF THE INVENTION

(S)-2-(3S,8S)-3-(4-(3,4-dichlorobenzyloxy)phenyl-7-((S)-1-phenylpropyl)-2,3,6,7,8,9-hexahydro-[1,4]-dioxino[2,3-g]isoquinolin-8-ylformylamino)-3-(4-(2,3-dimethylpyridin-4-yl)phenyl)propionic acid dihydrochloride (referred to herein as OAD2 dihydrochloride), is an orally available, non-peptide glucagon-like peptide 1 receptor (GLP-1R) agonist. It has an empirical formula of C₅₀H₄₉Cl₄N₃O₆, a molecular weight of 929.76, and the following chemical structure:

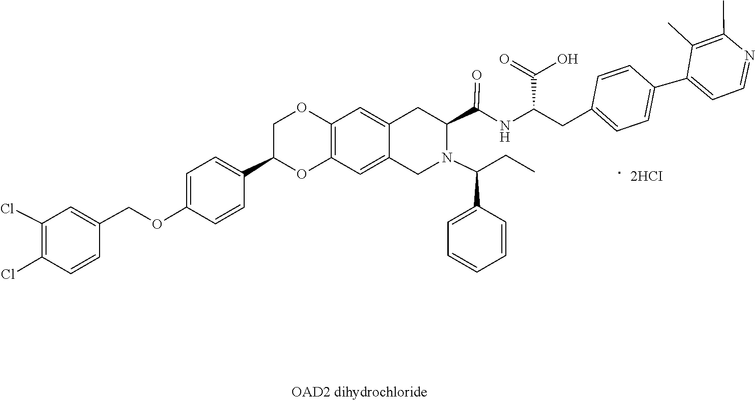 [SMILES: CC[C@@H](c1ccccc1)N1Cc2cc3c(cc2C[C@H]1C(=O)N[C@@H](Cc1ccc(-c2ccnc(C)c2C)cc1)C(=O)O)OC[C@H](c1ccc(OCc2ccc(Cl)c(Cl)c2)cc1)O3]

In addition to a patent for invention CN102378574B disclosing OAD2 dihydrochloride and the free base thereof, other acid salts of OAD2 or the crystal forms thereof have not been systematically studied. Therefore, it is desirable to further screen pharmaceutically acceptable acid salts of OAD2 and the crystal forms thereof.

### SUMMARY OF THE INVENTION

The objective of the invention is to provide pharmaceutically acceptable acid salts of OAD2: (S)-2-(3S,8S)-3-(4-(3,4-dichlorobenzyloxy)phenyl-7-((S)-1-phenylpropyl)-2,3,6,7,8,9-hexahydro-[1,4]-dioxino[2,3-g]isoquinolin-8-ylformylamino)-3-(4-(2,3-dimethylpyridin-4-yl)phenyl)propionic acid, and the preparation methods thereof.

To solve the above technical problems, the invention provides the following technical solutions:

The invention provides pharmaceutically acceptable acid salts of OAD2. In certain embodiments, the pharmaceutically acceptable acid salts of OAD2 may be crystalline. In another embodiment, the pharmaceutically acceptable salt of OAD2 is anhydrous, a hydrate, or a solvate. The invention also provides a method of making pharmaceutically acceptable acid salts of OAD2.

According to the invention, the crystalline acid salts of OAD2 include: crystal form B of hydrochloride, crystal form C of hydrochloride, crystal form C of p-toluenesulfonate, crystal form C of tartrate, crystal form C of citrate, crystal form C of glycollate, crystal form C of methanesulfonate, crystal form C of hydrobromide, crystal form D of hydrobromide, and crystal form C of sulfate.

The invention further provides a pharmaceutical composition comprising one or more pharmaceutically acceptable acid salt forms of OAD2. The invention also provides methods of producing a pharmaceutical composition comprising one or more pharmaceutically acceptable acid salt forms of OAD2.

The invention also provides use of a pharmaceutically acceptable acid salt of OAD2 for the preparation of a GLP-1 receptor agonist.

The invention also provides use of a pharmaceutically acceptable acid salt of OAD2 for the preparation of a medicament. In an embodiment, the medicament may be for treating diabetes.

The invention also provides methods of treatment comprising administering to a human in need thereof a therapeutically effective amount of a pharmaceutically acceptable acid salt of OAD2. The methods of treatment may be useful to treat a disorder or condition where activation of the GLP-1 receptor is beneficial.

These and other embodiments of the invention are described in greater detail in the detailed description of the invention which follows.

The pharmaceutically acceptable acid salts of OAD2 may achieve the following beneficial effects. Compared with OAD2 dihydrochloride, the pharmaceutically acceptable acid salts of OAD2 disclosed herein may have improved hygroscopicity. For example, but not limited to, crystal form B of hydrochloride, crystal form C of p-toluenesulfonate and crystal form C of sulfate show superior stability relative to OAD2 dihydrochloride.

### BRIEF DESCRIPTION OF THE FIGURES

FIG. 1 shows the XRPD of crystal form B of hydrochloride.

FIG. 2 shows the XRPD of crystal form C of hydrochloride.

FIG. 3 shows the XRPD of crystal form C of p-toluenesulfonate.

FIG. 4 shows the XRPD of crystal form C of tartrate.

FIG. 5 shows the XRPD of crystal form C of citrate.

FIG. 6 shows the XRPD of crystal form C of glycollate.

FIG. 7 shows the XRPD of crystal form C of methanesulfonate.

FIG. 8 shows the XRPD of crystal form C of hydrobromide.

FIG. 9 shows the XRPD of crystal form D of hydrobromide.

FIG. 10 shows the XRPD of crystal form C of sulfate.

FIG. 11 shows the TGA thermogram of crystal form B of hydrochloride.

FIG. 12 shows the DSC thermogram of crystal form B of hydrochloride.

FIG. 13 shows the TGA/DSC thermograms of crystal form C of hydrochloride.

FIG. 14 shows the TGA/DSC thermograms of crystal form C of p-toluenesulfonate.

FIG. 15 shows the TGA/DSC thermograms of crystal form C of tartrate.

FIG. 16 shows the TGA/DSC thermograms of crystal form C of citrate.

FIG. 17 shows the TGA/DSC thermograms of crystal form C of glycollate.

FIG. 18 shows the TGA/DSC thermograms of crystal form C of methanesulfonate.

FIG. 19 shows the TGA/DSC thermograms of crystal form C of hydrobromide.

FIG. 20 shows the TGA/DSC thermograms of crystal form D of hydrobromide.

FIG. 21 shows the TGA/DSC thermograms of crystal form C of sulfate.

FIG. 22 shows the ¹H-NMR spectrum of crystal form C of p-toluenesulfonate.

FIG. 23 shows the ¹H-NMR spectrum of crystal form C of tartrate.

FIG. 24 shows the ¹H-NMR spectrum of crystal form C of citrate.

FIG. 25 shows the ¹H-NMR spectrum of crystal form C of glycollate.

FIG. 26 shows the ¹H-NMR spectrum of crystal form C of methanesulfonate.

### DETAILED DESCRIPTION OF THE INVENTION

The invention will be further illustrated by combining the following specific examples. The following examples are used to explain the method of the invention and the core concept thereof, and for those skilled in the art, any possible change or substitution without departing from the inventive concept will fall within the protection scope of the invention. In the following examples, where the specific conditions of the experimental methods are not indicated, they are typically the conventional conditions, or are those recommended by the raw material or commodity manufactures; and the solvents without indicating the source are typically conventional solvents that are commercially available.

In the invention, “crystals” or “crystal form” is identified by the characterization of the indicated X-ray powder diffraction pattern. Those skilled in the art will appreciate that the experimental error of the characterization data typically depends on the conditions of the instrument, the preparation and purity of the sample, etc. Particularly, it is well known to those skilled in the art that X-ray powder diffraction pattern usually changes with the variation of experimental conditions, and accordingly the peak intensity per se cannot be identified as the only or decisive factor. The experimental error of peak angle is usually within 5% or less, and the data of peak angle usually allows for an error of ±0.2. In addition, owning to the influence of experimental factors such as the height of the sample, the peak angles may shift as a whole, and thus a certain shift is usually allowed. Those skilled in the art will appreciate that any crystal form having characteristic peaks same as or similar to those as shown in the X-ray powder diffraction pattern of the invention will fall within the protection scope of the invention. The value of the melting point as illustrated in the DSC thermogram should be interpreted as a value within a range of that numerical ±3.0° C., and preferably within a range of that numerical ±1.0° C.

The term “therapeutically effective amount” is used herein to denote the amount of the pharmaceutically acceptable acid salt of OAD2 that will elicit the therapeutic response of a subject that is being sought. In an embodiment, the therapeutic response may be agonizing the GLP-1 receptor.

OAD2 as a free base may be obtained according to the method as described in patent CN102378574B or in the related international publication WO 2010/114824, which are incorporated herein by reference in their entirety.

### Pharmaceutically Acceptable Salts of OAD2

The invention provides pharmaceutically acceptable acid salts of (S)-2-(3S,8S)-3-(4-(3,4-dichlorobenzyloxy)phenyl-7-((S)-1-phenylpropyl)-2,3,6,7,8,9-hexahydro-[1,4]-dioxino[2,3-g]isoquinolin-8-ylformylamino)-3-(4-(2,3-dimethylpyridin-4-yl)phenyl)propionic acid (“OAD2”).

In one embodiment, the present invention is a pharmaceutically acceptable acid salt formed between OAD2 and a pharmaceutically acceptable acid. In one embodiment, the pharmaceutically acceptable acid is selected from the group consisting of 1-hydroxy-2-naphthoic acid, 4-aminosalicylic acid, adipic acid, L-aspartic acid, benzene sulfonic acid, benzoic acid, trans-cinnamic acid, citric acid, fumaric acid, galactaric acid, gentisic acid, gluconic acid, glutamic acid, glutaric acid, glycolic acid, hexanoic acid, hippuric acid, hydrobromic acid, hydrochloric acid, L-lactic acid, maleic acid, L-malic acid, malonic acid, R-mandelic acid, methane sulfonic acid, naphthalene sulfonic acid, nicotinic acid, oxalic acid, palmitic acid, phosphoric acid, propionic acid, saccharin, salicyclic acid, stearic acid, succinic acid, sulfuric acid, L-tartaric acid, p-toluenesulfonic acid, vanillic acid, and vanillin. In one embodiment, the pharmaceutically acceptable acid is selected from the group consisting of hydrobromic acid, hydrochloric acid, p-toluenesulfonic acid, tartaric acid, citric acid, glycolic acid, methanesulfonic acid, and sulfonic acid.

In certain embodiments, the pharmaceutically acceptable acid salts of OAD2 may be crystalline. In another embodiment, the pharmaceutically acceptable acid salt of OAD2 is anhydrous, a hydrate, or a solvate.

According to the invention, the crystalline acid salts of OAD2 include: crystal form B of hydrochloride, crystal form C of hydrochloride, crystal form C of p-toluenesulfonate, crystal form C of tartrate, crystal form C of citrate, crystal form C of glycollate, crystal form C of methanesulfonate, crystal form C of hydrobromide, crystal form D of hydrobromide, and crystal form C of sulfate.

As a specific embodiment, in the above crystalline acid salts of OAD2, the salt formation ratio (molar ratio) is in the range of acid:free base=0.3:1˜1:1, but is not limited to that range, as the salt formation ratio (molar ratio) is in connection with the amount of the acid.

### Hydrochloride

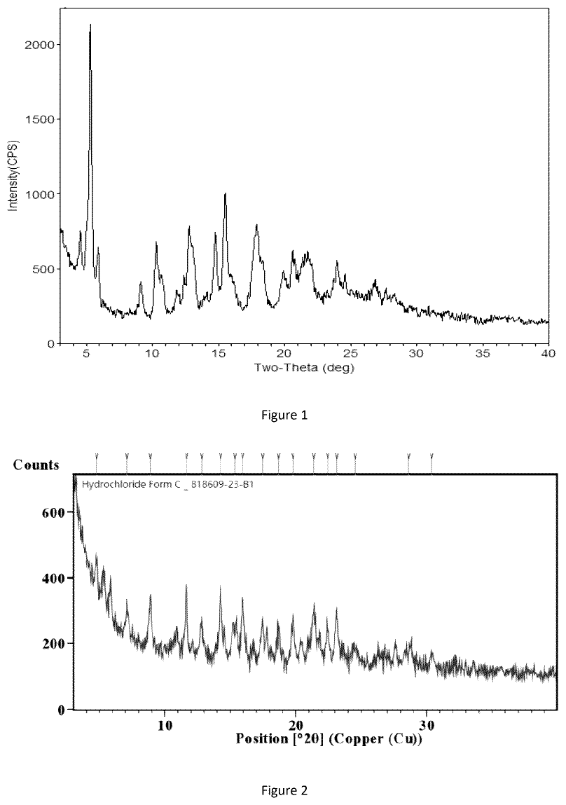

As a specific embodiment, the invention provides a crystal form B of hydrochloride of OAD2, which has an X-ray powder diffraction pattern comprising peaks at the following diffraction angles (2θ): 5.3±0.2°, 9.2±0.2°, 10.3±0.2°, 13.2±0.2°, and 14.8±0.2°; and preferably, the X-ray powder diffraction pattern comprises substantially the same peaks at the 2θ diffraction angles as shown in FIG. 1, and the X-ray powder diffraction data is as shown in Table 1.

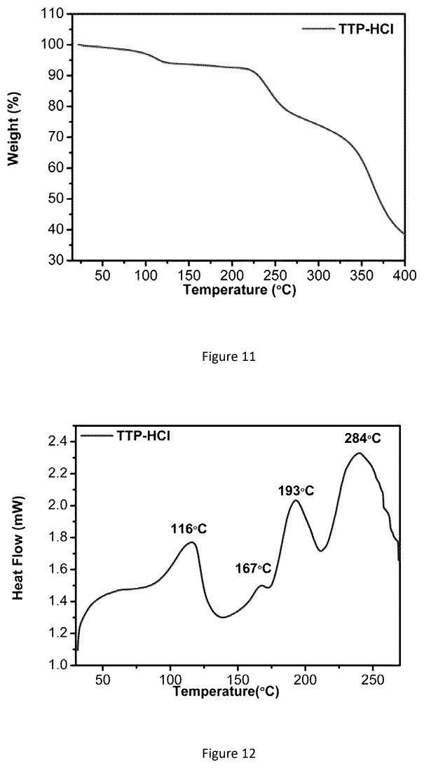

In another embodiment, the crystal form B of hydrochloride of OAD2 is characterized by an ¹H NMR spectrum. In another embodiment, the crystal form B of hydrochloride of OAD2 is characterized by an endothermic peak at 116° C. and/or 193° C. as determined by DSC. In another embodiment, the crystal form B of hydrochloride of OAD2 is characterized by a DSC profile as showing in FIG. 12. In another embodiment, the crystal form B of hydrochloride of OAD2 is characterized by a TGA profile as shown in FIG. 11. In another embodiment, the crystal form B of hydrochloride of OAD2 is characterized by at least two of the following features:
i) an XRPD pattern comprising peaks at the following diffraction angles (2θ): 5.3±0.2°, 9.2±0.2°, 10.3±0.2°, 13.2±0.2°, and 14.8±0.2°;
ii) a DSC profile as shown in FIG. 12; or
iii) a TGA profile as shown in FIG. 11.

As a specific embodiment, the invention provides a crystal form C of hydrochloride of OAD2, which has an X-ray powder diffraction pattern comprising peaks at the following diffraction angles (2θ): 8.9±0.2°, 11.6±0.2°, 14.3±0.2°, 15.9±0.2°, 21.4±0.2°, and 23.1±0.2°; and preferably, the X-ray powder diffraction pattern comprises substantially the same peaks at the 2θ diffraction angles as shown in FIG. 2, and the X-ray powder diffraction data is as shown in Table 1.

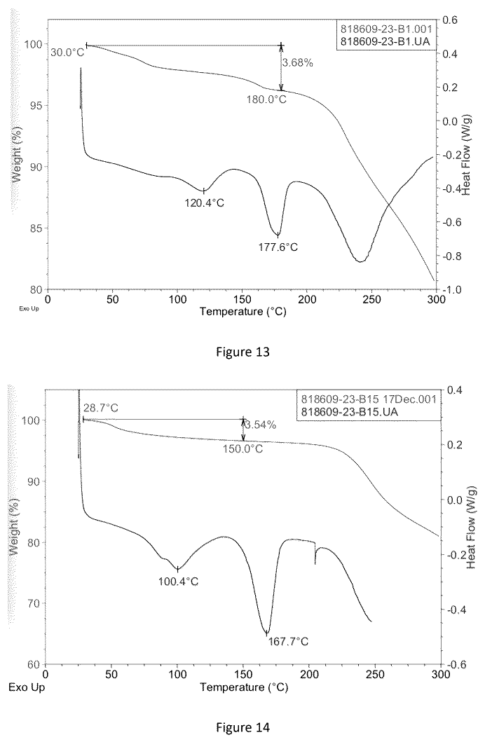

In another embodiment, the crystal form C of hydrochloride of OAD2 is characterized by an ¹H NMR spectrum. In another embodiment, the crystal form C of hydrochloride of OAD2 is characterized by an endothermic peak at 120.4° C. and/or 177.6° C. as determined by DSC. In another embodiment, the crystal form C of hydrochloride of OAD2 is characterized by a DSC profile as showing in FIG. 13. In another embodiment, the crystal form C of hydrochloride of OAD2 is characterized by a TGA profile as shown in FIG. 13. In another embodiment, the crystal form C of hydrochloride of OAD2 is characterized by at least two of the following features:
i) an XRPD pattern comprising peaks at the following diffraction angles (2θ): 8.9±0.2°, 11.6±0.2°, 14.3±0.2°, 15.9±0.2°, 21.4±0.2°, and 23.1±0.2°;
ii) a DSC profile as shown in FIG. 13; or
iii) a TGA profile as shown in FIG. 13.

### P-Toluenesulfonate

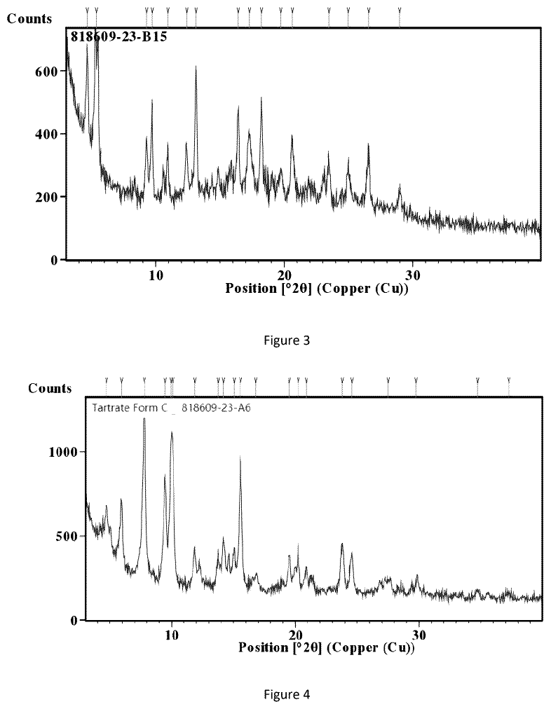

As a specific embodiment, the invention provides a crystal form C of p-toluenesulfonate of OAD2, which has an X-ray powder diffraction pattern comprising peaks at the following diffraction angles (2θ): 4.7±0.2°, 5.4±0.2°, 9.7±0.2°, 13.1±0.2°, 16.4±0.2° and 18.2±0.2°; and preferably, the X-ray powder diffraction pattern comprises substantially the same peaks at the 2θ diffraction angles as shown in FIG. 3, and the X-ray powder diffraction data is as shown in Table 1. In another embodiment, the crystal form C of p-toluenesulfonate of OAD2 is characterized by an ¹H NMR spectrum as shown in FIG. 22. In another embodiment, the crystal form C of p-toluenesulfonate of OAD2 is characterized by an endothermic peak at 100.4° C. and/or 167.7° C. as determined by DSC. In another embodiment, the crystal form C of p-toluenesulfonate of OAD2 is characterized by a DSC profile as showing in FIG. 14. In another embodiment, the crystal form C of p-toluenesulfonate of OAD2 is characterized by a TGA profile as shown in FIG. 14. In another embodiment, the crystal form C of p-toluenesulfonate of OAD2 is characterized by at least two of the following features:
i) an XRPD pattern comprising peaks at the following diffraction angles (2θ): 4.7 0.2°, 5.4±0.2°, 9.7±0.2°, 13.1±0.2°, 16.4±0.2° and 18.2±0.2°;
ii) a DSC profile as shown in FIG. 14;
iii) a TGA profile as shown in FIG. 14; or
iv) a ¹H NMR substantially similar to FIG. 22.

### Tartrate

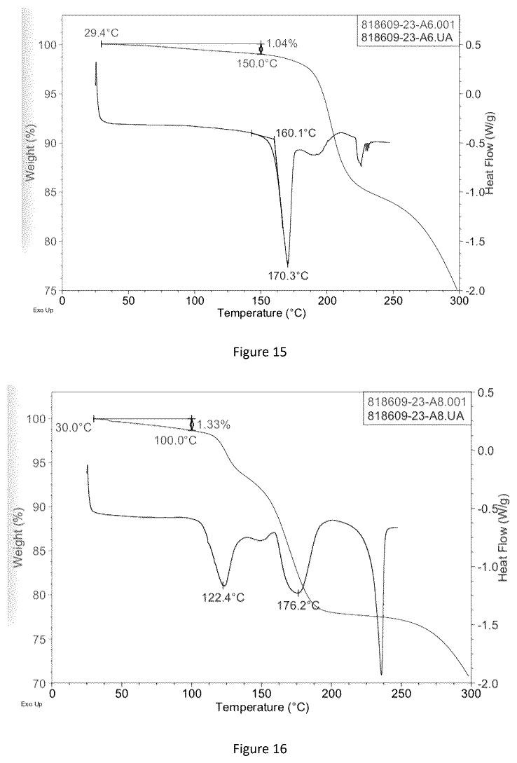

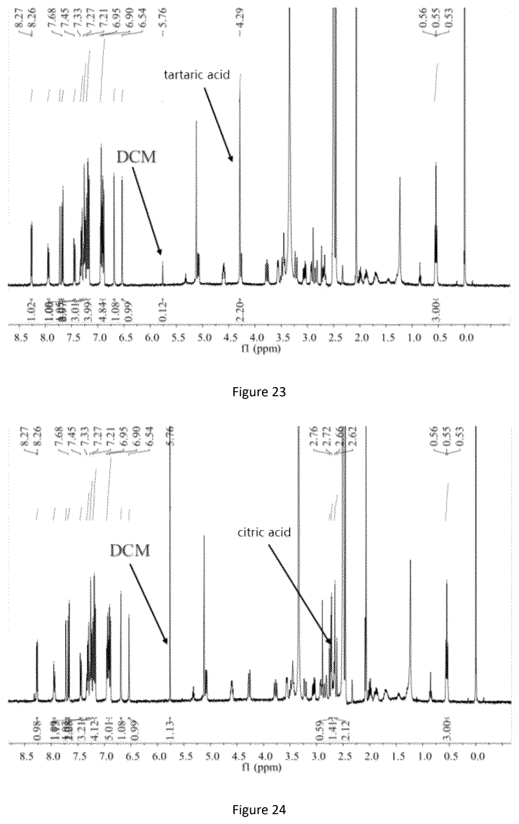

As a specific embodiment, the invention provides a crystal form C of tartrate of OAD2, which has an X-ray powder diffraction pattern comprising peaks at the following diffraction angles (2θ): 7.8±0.2°, 9.9±0.2°, 10.1±0.2°, and 15.6±0.2°; and preferably, the X-ray powder diffraction pattern comprises substantially the same peaks at the 2θ diffraction angles as shown in FIG. 4, and the X-ray powder diffraction data is as shown in Table 1. In another embodiment, the crystal form C of tartrate of OAD2 is characterized by an ¹H NMR spectrum as shown in FIG. 23. In another embodiment, the crystal form C of tartrate of OAD2 is characterized by an endothermic peak at 170.3° C. as determined by DSC. In another embodiment, the crystal form C of tartrate of OAD2 is characterized by a DSC profile as showing in FIG. 15. In another embodiment, the crystal form C of tartrate of OAD2 is characterized by a TGA profile as shown in FIG. 15. In another embodiment, the crystal form C of tartrate of OAD2 is characterized by at least two of the following features:
i) an XRPD pattern comprising peaks at the following diffraction angles (2θ): 7.8±0.2°, 9.9±0.2°, 10.1±0.2°, and 15.6±0.2°;
ii) a DSC profile as shown in FIG. 15;
iii) a TGA profile as shown in FIG. 15;
iv) a ¹H NMR substantially similar to 23.

### Citrate

As a specific embodiment, the invention provides a crystal form C of citrate of OAD2, which has an X-ray powder diffraction pattern comprising peaks at the following diffraction angles (2θ): 4.5±0.2°, 6.5±0.2°, and 15.9±0.2°; and preferably, the X-ray powder diffraction pattern comprises substantially the same peaks at the 2θ diffraction angles as shown in FIG. 5, and the X-ray powder diffraction data is as shown in Table 1. In another embodiment, the crystal form C of citrate of OAD2 is characterized by an ¹H NMR spectrum as shown in FIG. 24. In another embodiment, the crystal form C of citrate of OAD2 is characterized by an endothermic peak at 122.4° C. and/or 176.2° C. as determined by DSC. In another embodiment, the crystal form C of citrate of OAD2 is characterized by a DSC profile as showing in FIG. 16. In another embodiment, the crystal form C of citrate of OAD2 is characterized by a TGA profile as shown in FIG. 16. In another embodiment, the crystal form C of citrate of OAD2 is characterized by at least two of the following features: i) an XRPD pattern comprising peaks at the following diffraction angles (2θ): 4.5±0.2°, 6.5±0.2°, and 15.9±0.2°;
ii) a DSC profile as shown in FIG. 16;
iii) a TGA profile as shown in FIG. 16; or
iv) a ¹H NMR substantially similar to 24.

### Glycollate

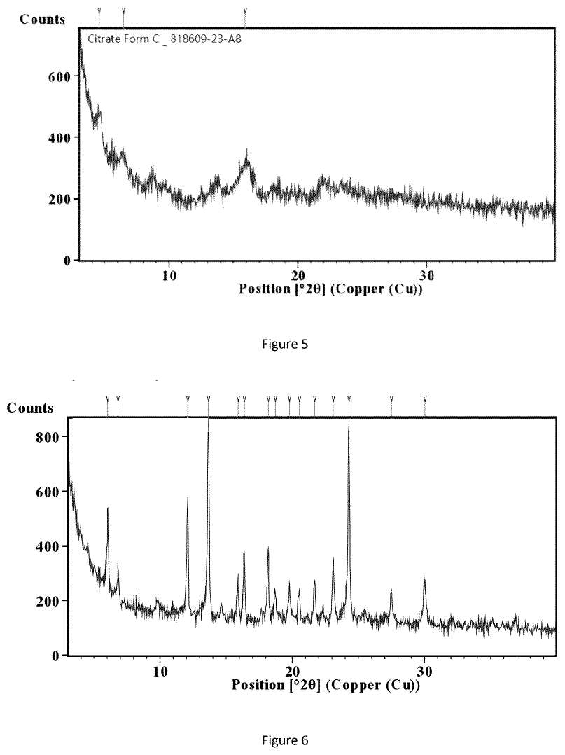

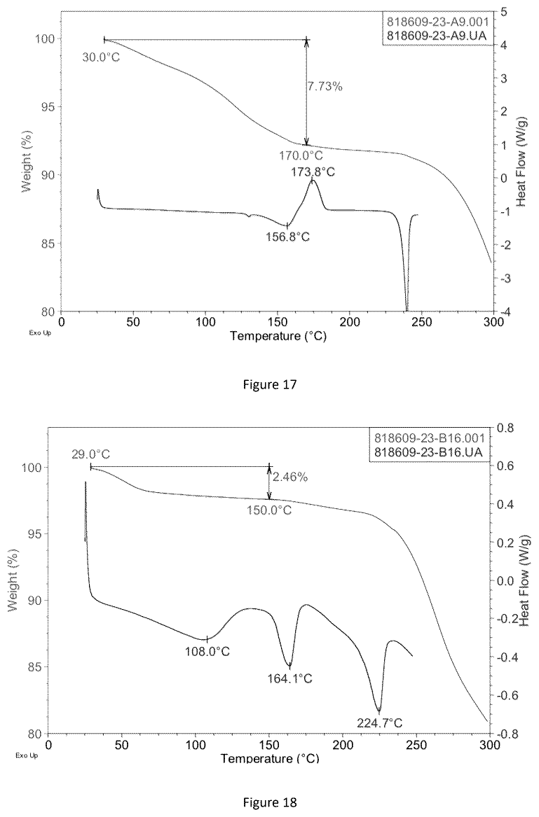

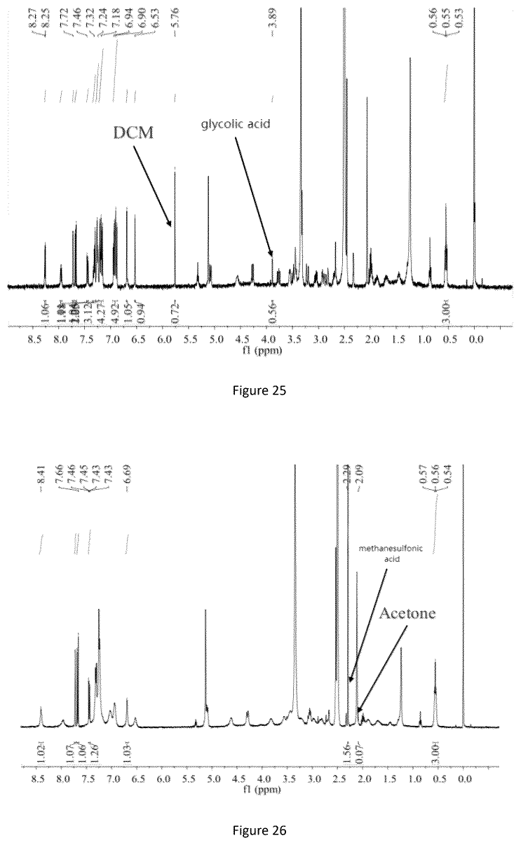

As a specific embodiment, the invention provides a crystal form C of glycollate of OAD2, which has an X-ray powder diffraction pattern comprising peaks at the following diffraction angles (2θ): 6.0±0.2°, 12.1±0.2°, 13.6±0.2°, 18.2±0.2° and 24.3±0.2°; and preferably, the X-ray powder diffraction pattern comprises substantially the same peaks at the 2θ diffraction angles as shown in FIG. 6, and the X-ray powder diffraction data is as shown in Table 1. In another embodiment, the crystal form C of glycollate of OAD2 is characterized by an ¹H NMR spectrum as shown in FIG. 25. In another embodiment, the crystal form C of glycollate of OAD2 is characterized by an endothermic peak at 156.8° C. and/or 173.8° C. as determined by DSC. In another embodiment, the crystal form C of glycollate of OAD2 is characterized by a DSC profile as showing in FIG. 17. In another embodiment, the crystal form C of glycollate of OAD2 is characterized by a TGA profile as shown in FIG. 17. In another embodiment, the crystal form C of glycollate of OAD2 is characterized by at least two of the following features:
i) an XRPD pattern comprising peaks at the following diffraction angles (2θ): 6.0±0.2°, 12.1±0.2°, 13.6±0.2°, 18.2±0.2° and 24.3±0.2°;
ii) a DSC profile as shown in FIG. 17;
iii) a TGA profile as shown in FIG. 17; or
iv) a ¹H NMR substantially similar to 25.

### Methanesulfonate

As a specific embodiment, the invention provides a crystal form C of methanesulfonate of OAD2, which has an X-ray powder diffraction pattern comprising peaks at the following diffraction angles (2θ): 10.2±0.2°, 12.8±0.2°, 13.8±0.2°, 14.4±0.2° and 22.2±0.2°; and preferably, the X-ray powder diffraction pattern comprises substantially the same peaks at the 2θ diffraction angles as shown in FIG. 7, and the X-ray powder diffraction data is as shown in Table 1. In another embodiment, the crystal form C of methanesulfonate of OAD2 is characterized by an ¹H NMR spectrum as shown in FIG. 26. In another embodiment, the crystal form C of methanesulfonate of OAD2 is characterized by an endothermic peak at 108.0, 164.1, and/or 224.7° C. as determined by DSC. In another embodiment, the crystal form C of methanesulfonate of OAD2 is characterized by a DSC profile as showing in FIG. 18. In another embodiment, the crystal form C of methanesulfonate of OAD2 is characterized by a TGA profile as shown in FIG. 18. In another embodiment, the crystal form C of methanesulfonate of OAD2 is characterized by at least two of the following features:
i) an XRPD pattern comprising peaks at the following diffraction angles (2θ): 10.2±0.2°, 12.8±0.2°, 13.8±0.2°, 14.4±0.2° and 22.2±0.2°;
ii) a DSC profile as shown in FIG. 18;
iii) a TGA profile as shown in FIG. 18; or
iv) a ¹H NMR substantially similar to 26.

### Hydrobromide

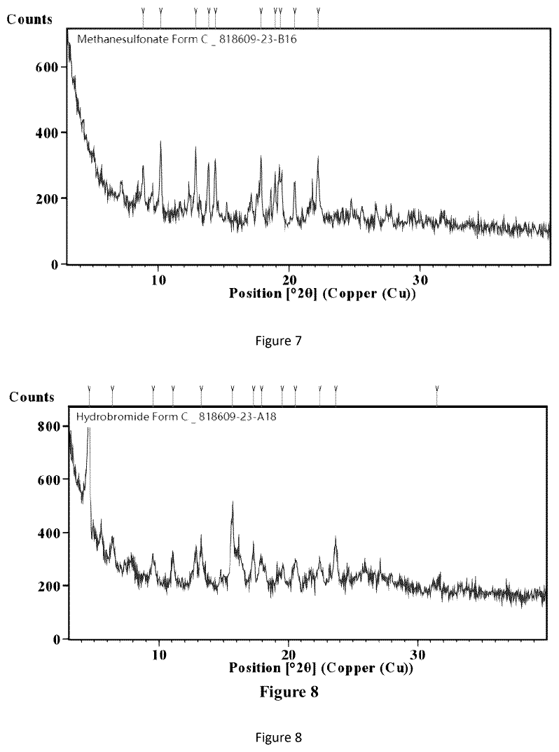

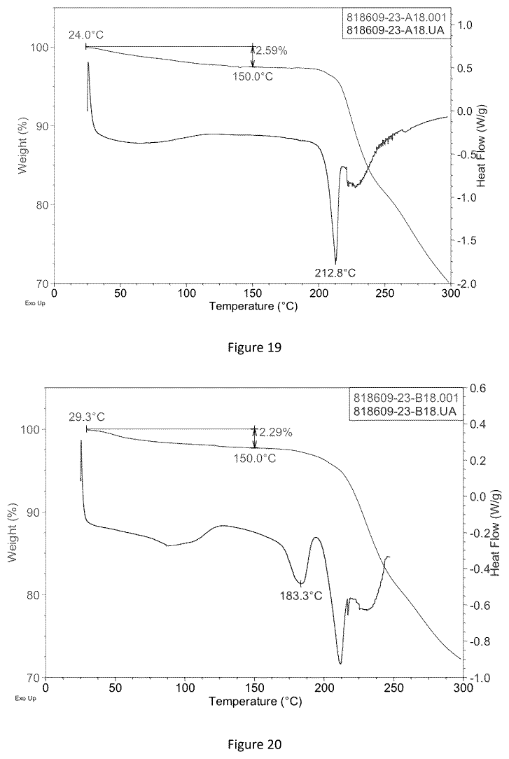

As a specific embodiment, the invention provides a crystal form C of hydrobromide of OAD2, which has an X-ray powder diffraction pattern comprising peaks at the following diffraction angles (2θ): 4.6±0.2°, 15.7±0.2°, and 23.7±0.2°; and preferably, the X-ray powder diffraction pattern comprises substantially the same peaks at the 2θ diffraction angles as shown in FIG. 8, and the X-ray powder diffraction data is as shown in Table 1. In another embodiment, the crystal form C of hydrobromide of OAD2 is characterized by an ¹H NMR spectrum. In another embodiment, the crystal form C of hydrobromide of OAD2 is characterized by an endothermic peak at 212.8° C. as determined by DSC. In another embodiment, the crystal form C of hydrobromide of OAD2 is characterized by a DSC profile as showing in FIG. 19. In another embodiment, the crystal form C of hydrobromide of OAD2 is characterized by a TGA profile as shown in FIG. 19. In another embodiment, the crystal form C of hydrobromide of OAD2 is characterized by at least two of the following features:
i) an XRPD pattern comprising peaks at the following diffraction angles (2θ): 4.6±0.2°, 15.7±0.2°, and 23.7±0.2°;
ii) a DSC profile as shown in FIG. 19; or
iii) a TGA profile as shown in FIG. 19.

As a specific embodiment, the invention provides a crystal form D of hydrobromide of OAD2, which has an X-ray powder diffraction pattern comprising peaks at the following diffraction angles (2θ): 4.7±0.2°, 8.8±0.2°, 15.9±0.2°, and 17.8±0.2°; and preferably, the X-ray powder diffraction pattern comprises substantially the same peaks at the 2θ diffraction angles as shown in FIG. 9, and the X-ray powder diffraction data is as shown in Table 1. In another embodiment, the crystal form D of hydrobromide of OAD2 is characterized by an ¹H NMR spectrum. In another embodiment, the crystal form D of hydrobromide of OAD2 is characterized by an endothermic peak at 183.3° C. as determined by DSC. In another embodiment, the crystal form D of hydrobromide of OAD2 is characterized by a DSC profile as showing in FIG. 20. In another embodiment, the crystal form D of hydrobromide of OAD2 is characterized by a TGA profile as shown in FIG. 20. In another embodiment, the crystal form D of hydrobromide of OAD2 is characterized by at least two of the following features:
i) an XRPD pattern comprising peaks at the following diffraction angles (2θ): 4.7±0.2°, 8.8±0.2°, 15.9±0.2°, and 17.8±0.2°;
ii) a DSC profile as shown in FIG. 20; or
iii) a TGA profile as shown in FIG. 20.

### Sulfate

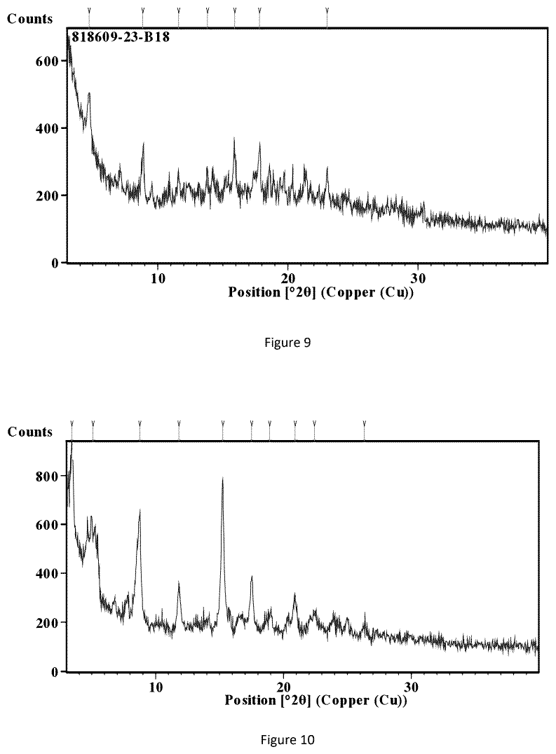

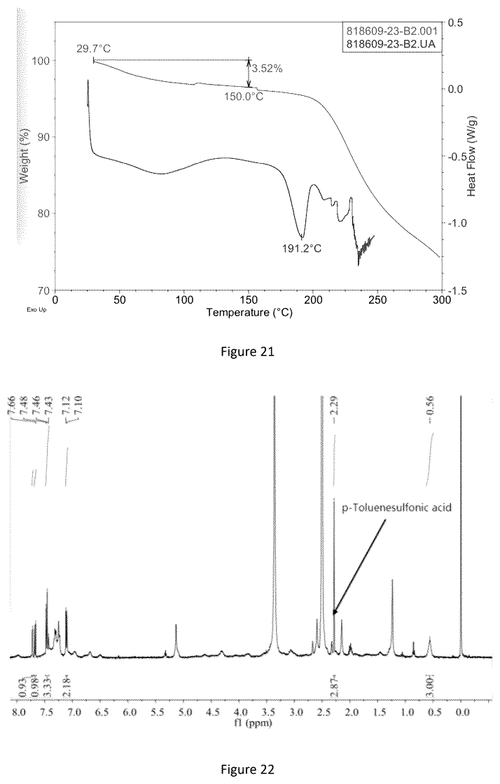

As a specific embodiment, the invention provides a crystal form C of sulfate of OAD2, which has an X-ray powder diffraction pattern comprising peaks at the following diffraction angles (2θ): 3.4±0.2°, 8.7±0.2°, and 15.2±0.2°; and preferably, the X-ray powder diffraction pattern comprises substantially the same peaks at the 2θ diffraction angles as shown in FIG. 10, and the X-ray powder diffraction data is as shown in Table 1. In another embodiment, the crystal form C of sulfate of OAD2 is characterized by an ¹H NMR spectrum. In another embodiment, the crystal form C of sulfate of OAD2 is characterized by an endothermic peak at 191.2° C. as determined by DSC. In another embodiment, the crystal form C of sulfate of OAD2 is characterized by a DSC profile as showing in FIG. 21. In another embodiment, the crystal form C of sulfate of OAD2 is characterized by a TGA profile as shown in FIG. 21. In another embodiment, the crystal form C of sulfate of OAD2 is characterized by at least two of the following features:
i) an XRPD pattern comprising peaks at the following diffraction angles (2θ): 3.4±0.2°, 8.7±0.2°, and 15.2±0.2°;
ii) a DSC profile as shown in FIG. 21; or
iii) a TGA profile as shown in FIG. 21.

TABLE 1
X-ray diffraction data (2θ) of crystalline acid salts of OAD2

| Crystal form | 2θ (±0.2°) | | | | | | |
|---|---|---|---|---|---|---|---|
| Crystal form B of | 4.5 | 5.3 | 5.9 | 9.2 | 10.3 | 10.7 | 12.0 |
| hydrochloride | 12.4 | 12.9 | 13.2 | 14.8 | 15.5 | 15.9 | 17.8 |
| | 19.9 | 20.6 | 21.7 | 24.0 | 24.6 | 26.7 | 27.2 |
| Crystal form C of | 4.8 | 7.1 | 8.9 | 11.6 | 12.8 | 14.3 | 15.3 |
| hydrochloride | 15.9 | 17.5 | 18.7 | 19.8 | 21.2 | 22.4 | 23.1 |
| | 24.5 | 28.6 | 30.4 | / | / | / | / |
| Crystal form C of p- | 4.7 | 5.4 | 9.3 | 9.7 | 10.9 | 12.4 | 13.1 |
| toluenesulfonate | 16.4 | 17.3 | 18.2 | 19.7 | 20.6 | 23.5 | 25.0 |
| | 25.6 | 29.0 | / | / | / | / | / |
| Crystal form C of | 4.7 | 5.9 | 7.8 | 9.5 | 9.9 | 10.1 | 11.9 |
| tartrate | 13.7 | 14.2 | 15.0 | 15.6 | 16.8 | 19.5 | 20.2 |
| | 20.9 | 23.8 | 24.6 | 27.5 | 29.8 | / | / |
| Crystal form C of | 4.5 | 6.5 | 15.9 | / | / | / | / |
| citrate | | | | | | | |
| Crystal form C of | 6.0 | 6.8 | 12.1 | 13.6 | 15.9 | 16.4 | 18.2 |
| glycollate | 18.7 | 19.8 | 20.5 | 21.7 | 23.1 | 24.3 | 27.5 |
| | 30.0 | / | / | / | / | / | / |
| Crystal form C of | 8.8 | 10.2 | 12.8 | 13.8 | 14.4 | 17.8 | 18.9 |
| methanesulfonate | 19.3 | 20.4 | 22.2 | / | / | / | / |
| Crystal form C of | 4.6 | 6.4 | 9.6 | 11.1 | 13.3 | 15.7 | 17.3 |
| hydrobromide | 17.9 | 19.5 | 20.5 | 22.4 | 23.7 | / | / |
| Crystal form D of | 4.7 | 8.8 | 11.6 | 13.8 | 15.9 | 17.8 | 23.0 |
| hydrobromide | | | | | | | |
| Crystal form C of | 3.4 | 5.1 | 8.7 | 11.8 | 15.2 | 17.5 | 18.9 |
| sulfate | 20.9 | 22.4 | 26.3 | / | / | / | / |

### Pharmaceutical Compositions

The invention further provides a pharmaceutical composition comprising one or more pharmaceutically acceptable acid salt forms of OAD2, and optionally a pharmaceutical acceptable carrier. In another embodiment, the invention provides a pharmaceutical composition comprising a crystalline, pharmaceutically acceptable acid salt of OAD2, wherein the pharmaceutical composition comprises a therapeutically effective amount of the crystalline, pharmaceutically acceptable acid salt of OAD2, and optionally a pharmaceutical acceptable carrier.

In another embodiment, the pharmaceutical composition of the invention comprises a pharmaceutically acceptable acid salt formed between OAD2 and a pharmaceutically acceptable acid. In one embodiment, the pharmaceutically acceptable acid is selected from the group consisting of 1-hydroxy-2-naphthoic acid, 4-aminosalicylic acid, adipic acid, L-aspartic acid, benzene sulfonic acid, benzoic acid, trans-cinnamic acid, citric acid, fumaric acid, galactaric acid, gentisic acid, gluconic acid, glutamic acid, glutaric acid, glycolic acid, hexanoic acid, hippuric acid, hydrobromic acid, hydrochloric acid, L-lactic acid, maleic acid, L-malic acid, malonic acid, R-mandelic acid, methane sulfonic acid, naphthalene sulfonic acid, nicotinic acid, oxalic acid, palmitic acid, phosphoric acid, propionic acid, saccharin, salicyclic acid, stearic acid, succinic acid, sulfuric acid, L-tartaric acid, p-toluenesulfonic acid, vanillic acid, and vanillin. In another embodiment, the pharmaceutically acceptable acid is selected from the group consisting of hydrobromic acid, hydrochloric acid, p-toluenesulfonic acid, tartaric acid, citric acid, glycolic acid, methanesulfonic acid, and sulfonic acid.

The pharmaceutical composition may be prepared according to methods known in the art. Preferably, the crystalline acid salts of OAD2 of the invention is 0.001%˜99% by weight of the pharmaceutical composition. As a specific embodiment, the crystalline acid salt of OAD2 is combined with one or more pharmaceutically acceptable carriers and formulated into any dosage form that is suitable for use in human or non-human animals.

Pharmaceutical compositions of the present invention comprising a pharmaceutically acceptable acid salt of OAD2 may be in a form suitable for oral use, for example, as tablets, troches, lozenges, dispersible powders or granules, or hard or soft capsules. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations.

Tablets, tronches, lozenges, dispersible powders or granules, or hard or soft capsules may contain a pharmaceutically acceptable acid salt of OAD2 in admixture with one or more pharmaceutically-acceptable carriers which are suitable for the manufacture of such tablets, tronches, lozenges, dispersible powders or granules, or hard or soft capsules.

A “pharmaceutically acceptable carrier” is a medium generally accepted in the art for the delivery of biologically active agents to mammals, e.g., humans. Such carriers are generally formulated according to a number of factors well within the purview of those of ordinary skill in the art to determine and account for. These include, without limitation, the type and nature of the active agent being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art. Descriptions of suitable pharmaceutically acceptable carriers, and factors involved in their selection, are found in a variety of readily available sources.

### Methods of Treatment

In another aspect, the present invention provides pharmaceutical compositions comprising a therapeutically effective amount of a pharmaceutically acceptable acid salt of OAD2 wherein a therapeutically effective amount of a pharmaceutically acceptable acid salt OAD2 comprises a sufficient amount for the treatment of a condition or disorder where activation of the GLP-1 receptor is beneficial.

In another aspect, the present invention also provides a method of treatment comprising administering a therapeutically effective amount of a pharmaceutically acceptable salt of OAD2 to a human in need thereof. The method may comprise administering a pharmaceutical composition comprising a therapeutically effective amount of a pharmaceutically acceptable acid salt of OAD2 to a human in need thereof. In another embodiment, the pharmaceutically acceptable salt of OAD2 may be administered in crystalline form.

The methods of treatment may useful to treat a disorder or condition where activation of the GLP-1 receptor is beneficial, such as, but not limited to a disorder or condition is selected from the group consisting of: metabolic syndrome, glucose intolerance, hyperglycemia, dyslipidemia, diabetes mellitus type 1, diabetes mellitus type 2, hypertriglyceridemia, syndrome X, insulin resistance, impaired glucose tolerance (IGT), obesity, diabetic dyslipidemia, hyperlipidemia, arteriosclerosis, atherosclerosis, other cardiovascular diseases, hypertension, metabolic disorders that where activation of the GLP-1 receptor is beneficial, and complications resulting from or associated with diabetes, including, but not limited to, neuropathy, retinopathy, nephropathy, and impaired wound healing. In an embodiment, the condition treated is type 2 diabetes.

A pharmaceutically acceptable acid salt of OAD2 of the present invention may be administered at a dosage level such that the amount of OAD2 administered is between 1 mg and 100 mg per day. The dosage may be individualized by the clinician based on the specific clinical condition of the subject being treated. Thus, it will be understood that the specific dosage level for any particular subject will depend upon a variety of factors including the activity of the specific compound employed, the age, body weight, general health, sex, diet, time of administration, route of administration, rate of excretion, drug combination and the severity of the particular disease undergoing therapy.

### EXAMPLES

### Experimental Instruments and Protocols

1. X-ray powder dffraction (XRPD): See the table 2 below showing the test parameters.

TABLE 2
XRPD test parameters

| Parameters | Instrument 1 | Instrument 2 | Instrument 3 |
|---|---|---|---|
| Model | Empyrean | X′ Pert3 | X′ Pert3 |
| X-ray | Cu, Kα, | Cu, Kα, | Cu, Kα, |
| | Kα1 (Å: 1.540598, | Kα1 (Å: 1.540598, | Kα1 (Å: 1.540598, |
| | Kα2 (Å: 1.544426 | Kα2 (Å: 1.544426 | Kα2 (Å: 1.544426 |
| | Kα2/Kα1 intensity | Kα2/Kα1 intensity | Kα2/Kα1 intensity |
| | ratio: 0.50 | ratio: 0.50 | ratio: 0.50 |
| X-ray tube | 45 kV, 40 mA | 45 kV, 40 mA | 45 kV, 40 mA |
| settings | | | |
| Divergence | Automatic | ⅛° | ⅛° |
| slit | | | |
| Scan mode | Continuous | Continuous | Continuous |
| Scan range | 3-40 | 3-40 | 3-40 |
| (°2Theta) | | | |
| Scan time | 17.8 | 46.7 | 46.7 |
| per step (s) | | | |
| Scan step | 0.0167 | 0.0263 | 0.0263 |
| (°2Theta) | | | |
| Measure | ~5 min 30 s | ~5 min | ~5 min |
| time | | | |

2. Thermogravimetric analysis (TGA) and differential scanning calorimetry (DSC) Collected on TA Q5000/5500 thermogravimetric analyzer and TA Q200/Q2000/2500 differential scanning calorimeter, respectively. See the table 3 below showing the test parameters.

TABLE 3
DSC and TGA test parameters

| Parameters | TGA | DSC |
|---|---|---|
| Method | Linear heating | Linear heating |
| Sample | Aluminum plate, open | Aluminum plate, covered/ |
| | | uncovered |
| Temperature | Room temperature- | 25° C.-endpoint |
| | endpoint temperature | temperature |
| Scan rate (° C./min) | 10 | 10 |
| Gas protection | Nitrogen | Nitrogrn |

3. The liquid NMR was collected on Bruker 400M nuclear magnetic resonance with DMSO-d6 as the solvent.
4. The salt formation molar ratio test of ions is performed by ion chromatography. The test conditions are shown in Table 4.

TABLE 4
Salt formation molar ratio test conditions

| Ion chromatograph | ThermoFisher ICS-1100 |
|---|---|
| Chromatographic column | IonPac AS18 Analytical Column, 250 * 4 |
| | mm (anion) |
| | Dionex IonpacTM CS12A RFICTM, |
| | 250*4 mm (cation) |
| Mobile phase | 25 mM NaOH ((anion), 20 |
| | mMMethanesulfonic acid (cation) |
| Sample volume | 25 μL |
| Speed | 1.0 mL/min |
| System temperature | 35° |
| Column | 35° |
| Electric current | 80 mA |
| Running time | Chloride ion 6.0 min, Sulfate ion 10.0 |
| | min, Bromide ion 9.0 min |

5. Dynamic moisture adsorption (DVS):
   Dynamic moisture adsorption (DVS) curves were collected on DVS Intrinsic of Surface Measurement Systems (SMS). The relative humidity at 25° C. was corrected with the deliquescent points of LiCl, Mg (NO3) 2 and KCl. DVS test parameters are listed in Table 5.

TABLE 5
DVS test parameters

| Parameters | Settings |
|---|---|
| Temperature | 25° C. |
| Sample | 10-20 mg |
| Gas protection and flow speed | N2, 200 mL/min |
| dm/dt | 0.002%/min |
| Min dm/dt balance time | 10 min |
| Max balance time | 180 min |
| RH range | 0% RH-95% RH |
| RH gradient | 10%(0% RH-90% RH, 90% RH- |
| | 0% RH) |
| | 5%(90% RH-95% RH, 95% RH- |
| | 90% RH) |

### Example 1: Preparation of Crystal Form B of Hydrochloride

At 20° C., 1 g OAD2 was added to 5 ml 2-methyltetrahydrofuran, and then 1.4 ml of 1 mol/L hydrochloric acid solution was dropwise added. The mixture was stirred for 2 hours. XRPD was used to monitor whether a new crystal form was formed. After the completion of transformation and crystallization, crystal form B of hydrochloride was obtained after suction filtration and drying at 50° C., with a salt acid to base formation molar ratio of 1:1.

### Example 2: Preparation of Crystal Form C of Hydrochloride

0.3 g OAD2 was dissolved in an acetone/water solution containing hydrochloric acid (29.2 μl), and the mixture was stirred at room temperature. XRPD was used to monitor whether the solid had transformed into crystals. After the completion of transformation, crystal form C of hydrochloride was obtained after centrifugation and dryness, with a salt acid to base formation molar ratio of 1:1.

### Examples 3 to 12: Screening of Other Crystalline Salt Complexes and the Preparation Process Thereof

According to the method of Example 1 or 2, other crystalline acid salts were prepared, and the specific results were shown in Table 6. The results showed that, not all acids whose ΔpKa met the requirements could form salt with the free base; moreover, when the acid and the free base were fed at a ratio of 1:1, they might not always form a 1:1 salt.

TABLE 6
Investigation of acid salts

| | | | | | Salt |
|---|---|---|---|---|---|
| | | | | | formation |
| | Crystalline | | | Reference | molar ratio |
| Example | complex | Solvent | Acid | method | (acid/base) |
| 3 | Crystal form C of | tetrahydrofuran | p- | Example 1 | 1:1 |
| | P- | | toluenesulfonic | | |
| | toluenesulfonate | | acid | | |
| 4 | Crystal form C of | dichloromethane | tartaric acid | Example 2 | 1:1 |
| | tartrate | | | | |
| 5 | Crystal form C of | dichloromethane | citric acid | Example 2 | 1:1 |
| | citrate | | | | |
| 6 | Crystal form C of | dichloromethane | glycollic acid | Example 2 | 0.3:1 |
| | glycollate | | | | |
| 7 | Crystal form C of | 2-methyl- | methanesulfonic | Example 2 | 0.5:1 |
| | methanesulfonate | tetrahydrofuran | acid | | |
| 8 | Crystal form C of | dichloromethane | hydrobromic | Example 2 | 1:1 |
| | hydrobromide | | acid | | |
| 9 | Crystal form D of | 2-methyl- | hydrobromic | Example 2 | 1:1 |
| | hydrobromide | tetrahydrofuran | acid | | |
| 10 | Crystal form C of | 95% acetone- | sulfuric acid | Example 2 | 1:1 |
| | sulfate | water solution | | | |
| 11 | / | dichloromethane | phosphoric acid | Example 2 | No acid |
| | | or 95% acetone- | | | formed |
| | | water solution | | | |
| 12 | / | dichloromethane | fumaric acid | Example 2 | No acid |
| | | or 95% acetone- | | | formed |
| | | water solution | | | |

### Example 13: Experimental Characterization of Crystalline Salt Complexes

The solid crystal forms were characterized by means of XRPD, DSC/TGA, etc., and the experimental data was shown in Table 7.

TABLE 7
Characterization data of the crystal forms

| | | DSC | Molar |
|---|---|---|---|
| | TGA weight loss | endothermic | ratio |
| | (%, (temperature/ | peak (°, peak | (acid/ |
| Crystalline salt | ° C.)) | temperature) | base) |
| Crystal form B of | 6.3 (150) | 116, 193 | 1.0 |
| hydrochloride | | | |
| Crystal form C of | 3.7 (180) | 120.4, 177.6 | 1.0 |
| hydrochloride | | | |
| Crystal form C of sulfate | 3.5 (150) | 191.2 | 1.0 |
| Crystal form C of tartrate | 1.0 (150) | 170.3 | 1.0 |
| Crystal form C of citrate | 1.3 (100) | 122.4, 176.2 | 1.0 |
| Crystal form C of | 7.7 (170) | 156.8, 173.8 | 0.3 |
| glycollate | | | |
| Crystal form C of p- | 3.5 (150) | 100.4, 167.7 | 1.0 |
| toluenesulfonate | | | |
| Crystal form C of | 2.5 (170) | 108.0, 164.1, | 0.5 |
| methanesulfonate | | 224.7 | |
| Crystal form C of | 2.6 (150) | 212.8 | 1.0 |
| hydrobromide | | | |
| Crystal form D of | 2.3 (150) | 183.3 | 1.0 |
| hydrobromide | | | |

### Example 14: Study of the Properties of Crystalline Salt Complexes

The Crystalline salts were subject to tests to determine and compare the pH solubility, hygroscopicity, and solid stability thereof.

### 1) Assessment of pH Solubility

The solubility of crystal form B of hydrochloride, crystal form C of p-toluenesulfonate, crystal form C of sulfate, and crystal form C of hydrobromide under different pH conditions were determined and compared, and the results were shown in Table 8. All the crystalline salts had low solubility in buffers of pH 3.0 and 5.0. In a buffer of pH 1.0, crystal form C of p-toluenesulfonate had significantly higher solubility than other crystalline salts.

TABLE 8
Comparison of solubility of various crystalline salts under different
pH conditions

| | Solubility (mg/mL) | | | | |
|---|---|---|---|---|---|
| Crystalline salt | pH 1.0 | pH 2.0 | pH 3.0 | pH 5.0 | pH 7.0 |
| Crystal form B of | 0.381 | 0.017 | 0.002 | 0.001 | 0.109 |
| hydrochloride | | | | | |
| Crystal form C of | 1.423 | 0.118 | 0.001 | 0.001 | 0.093 |
| p-toluenesulfonate | | | | | |
| Crystal form C of sulfate | 0.811 | 0.074 | 0.001 | 0.001 | 0.096 |
| Crystal form C of | 0.496 | 0.016 | 0.001 | 0.004 | 0.039 |
| hydrobromide | | | | | |

### 2) Study of Hygroscopicity

Five crystalline salts were subject to DVS test (25° C., 80% RH). The results demonstrated that all the samples showed no change of crystal form before and after the DVS test; except that the sulfate had hygroscopicity, all the the remaining samples had slight hygroscopicity; but the hygroscopicity was significantly improved as compared with OAD2 dihydrochloride. The results were shown in Table 9.

TABLE 9

| | Moisture | Change |
|---|---|---|
| | adsorption | of crystal |
| Crystalline salt | rate (%) | form, yes/no |
| Crystal form B of hydrochloride | 0.8 | No |
| Crystal form C of p-toluenesulfonate | 0.6 | No |
| Crystal form C of sulfate | 3.2 | No |
| Crystal form C of hydrobromide | 1.4 | No |
| OAD2 dihydrochloride | 8.3 | / |

### 3) Assessment of Solid Stability

The stability of crystal form B of hydrochloride, crystal form C of sulfate, crystal form C of p-toluenesulfonate and compound I was compared. After placement under a 40° C./100% RH condition for 1 week, physical and chemical stability was assessed by means of XRPD and HPLC, respectively, and the results were shown in Table 10. The results demonstrated that all the samples showed no distinct decrease in purity and no change of crystal form, and had superior stability; and particularly, crystal form C of p-toluenesulfonate showed the best stability.

TABLE 10
Comparison of stability of various crystalline salts

| | HPLC (area %) | | Change |
|---|---|---|---|
| | | 40° C./100% | of crystal |
| Crystalline salt | Initiation | RH/7 d | form, yes/no |
| Crystal form B of hydrochloride | 98.37 | 98.18 | No |
| Crystal form C of sulfate | 98.85 | 98.78 | No |
| Crystal form C of p- | 98.85 | 98.87 | No |
| toluenesulfonate | | | |
| compound I | 98.91 | 97.51 | / |

1.-12. (canceled)

13. A method of treating obesity in a subject in need thereof, comprising orally administering to the subject a pharmaceutical composition comprising a crystalline form of a salt of (S)-2-(3S,8S)-3-(4-(3,4-dichlorobenzyloxy)phenyl-7-((S)-1-phenylpropyl)-2,3,6,7,8,9-hexahydro-[1,4]-dioxino[2,3-g]isoquinolin-8-ylformylamino)-3-(4-(2,3-dimethylpyridin-4-yl)phenyl)propionic acid (OAD2), selected from the group consisting of the following:
crystal form B of hydrochloride salt of OAD2 (form B),
crystal form C of hydrochloride salt of OAD2 (form C),
crystal form C of p-toluenesulfonate salt of OAD2 (form C),
crystal form C of tartrate salt of OAD2 (form C),
crystal form C of citrate salt of OAD2 (form C),
crystal form C of glycollate salt of OAD2 (form C),
crystal form C of methanesulfonate salt of OAD2 (form C),
crystal form C of hydrobromide salt of OAD2 (form C),
crystal form D of hydrobromide salt of OAD2 (form D), and
crystal form C of sulfate salt of OAD2 (form C); and
a pharmaceutically acceptable carrier.

14. The method of claim 13, wherein the crystalline form is a crystalline form of the hydrochloride salt of OAD2 and is characterized as having an X-ray powder diffraction pattern comprising peaks at the following diffraction angles (2θ): 5.3±0.2°, 9.2±0.2°, 10.3±0.2°, 13.2±0.2°, and 14.8±0.2° as measured using Cu, Kα radiation (form B).

15. The method of claim 13, wherein the crystalline form is a crystalline form of the hydrochloride salt of OAD2 and is characterized as having an X-ray powder diffraction pattern comprising substantially the same peaks at the 2θ diffraction angles as shown in FIG. 1 (form B).

16. The method of claim 13, wherein the crystalline form is a crystalline form of the hydrochloride salt of OAD2 and is characterized as having an X-ray powder diffraction pattern comprising peaks at the following diffraction angles (2θ): 8.9±0.2°, 11.6±0.2°, 14.3±0.2°, 15.9±0.2°, 21.4±0.2°, and 23.1±0.2° as measured using Cu, Kα radiation (form C).

17. The method of claim 13, wherein the crystalline form is a crystalline form of the hydrochloride salt of OAD2 and is characterized as having an X-ray powder diffraction pattern comprising substantially the same peaks at the 2θ diffraction angles as shown in FIG. 2 (form C).

18. The method of claim 13, wherein the crystalline form is a crystalline form of the p-toluenesulfonate salt of OAD2 and is characterized as having an X-ray powder diffraction pattern comprising peaks at the following diffraction angles (2θ): 4.7±0.2°, 5.4±0.2°, 9.7±0.2°, 13.1±0.2°, 16.4±0.2°, and 18.2±0.2° as measured using Cu, Kα radiation (form C).

19. The method of claim 13, wherein the crystalline form is a crystalline form of the p-toluenesulfonate salt of OAD2 and is characterized as having an X-ray powder diffraction pattern comprising substantially the same peaks at the 2θ diffraction angles as shown in FIG. 3 (form C).

20. The method of claim 13, wherein the crystalline form is a crystalline form of the tartrate salt of OAD2 and is characterized as having an X-ray powder diffraction pattern comprising peaks at the following diffraction angles (2θ): 7.8±0.2°, 9.9±0.2°, 10.1±0.2°, and 15.6±0.2° as measured using Cu, Kα radiation (form C).

21. The method of claim 13, wherein the crystalline form is a crystalline form of the tartrate salt of OAD2 and is characterized as having an X-ray powder diffraction pattern comprises substantially the same peaks at the 2θ diffraction angles as shown in FIG. 4 (form C).

22. The method of claim 13, wherein the crystalline form is a crystalline form of the citrate salt of OAD2 and is characterized as having an X-ray powder diffraction pattern comprising peaks at the following diffraction angles (2θ): 4.5±0.2°, 6.5±0.2°, and 15.9±0.2° as measured using Cu, Kα radiation (form C).

23. The method of claim 13, wherein the crystalline form is a crystalline form of the p-toluenesulfonate salt of OAD2 and is characterized as having an X-ray powder diffraction pattern comprising substantially the same peaks at the 2θ diffraction angles as shown in FIG. 5 (form C).

24. The method of claim 13, wherein the crystalline form is a crystalline form of the glycollate salt of OAD2 and is characterized as having an X-ray powder diffraction pattern comprising peaks at the following diffraction angles (2θ): 6.0±0.2°, 12.1±0.2°, 13.6±0.2°, 18.2±0.2°, and 24.3±0.2° as measured using Cu, Kα radiation (form C).

25. The method of claim 13, wherein the crystalline form is a crystalline form of the glycollate salt of OAD2 and is characterized as having an X-ray powder diffraction pattern comprising substantially the same peaks at the 2θ diffraction angles as shown in FIG. 6 (form C).

26. The method of claim 13, wherein the crystalline form is a crystalline form of the methanesulfonate salt of OAD2 and is characterized as having an X-ray powder diffraction pattern comprising peaks at the following diffraction angles (2θ): 10.2±0.2°, 12.8±0.2°, 13.8±0.2°, 14.4±0.2°, and 22.2±0.2° as measured using Cu, Kα radiation (form C).

27. The method of claim 13, wherein the crystalline form is a crystalline form of the methanesulfonate salt of OAD2 and is characterized as having an X-ray powder diffraction pattern comprising substantially the same peaks at the 2θ diffraction angles as shown in FIG. 7 (form C).

28. The method of claim 13, wherein the crystalline form is a crystalline form of the hydrobromide salt of OAD2 and is characterized as having an X-ray powder diffraction pattern comprising peaks at the following diffraction angles (2θ): 4.6±0.2°, 15.7±0.2°, and 23.7±0.2° as measured using Cu, Kα radiation (form C).

29. The method of claim 13, wherein the crystalline form is a crystalline form of the hydrobromide salt of OAD2 and is characterized as having an X-ray powder diffraction pattern comprising substantially the same peaks at the 2θ diffraction angles as shown in FIG. 8 (form C).

30. The method of claim 13, wherein the crystalline form is a crystalline form of the hydrobromide salt of OAD2 and is characterized as having an X-ray powder diffraction pattern comprising peaks at the following diffraction angles (2θ): 4.7±0.2°, 8.8±0.2°, 15.9±0.2°, and 17.8±0.2° (form D).

31. The method of claim 13, wherein the crystalline form is a crystalline form of the hydrobromide salt of OAD2 and is characterized as having an X-ray powder diffraction pattern comprising substantially the same peaks at the 2θ diffraction angles as shown in FIG. 9 (form D).

32. The method of claim 13, wherein the crystalline form is a crystalline form of the sulfate salt of OAD2 and is characterized as having an X-ray powder diffraction pattern comprising peaks at the following diffraction angles (2θ): 3.4±0.2°, 8.7±0.2°, and 15.2±0.2° as measured using Cu, Kα radiation (form C).

33. The method of claim 13, wherein the crystalline form is a crystalline form of the sulfate salt of OAD2 and is characterized as having an X-ray powder diffraction pattern comprising substantially the same peaks at the 2θ diffraction angles as shown in FIG. 10 (form C).

34. The method of claim 13, wherein the pharmaceutical composition is formulated as a tablet.

35. The method of claim 13, wherein the subject is administered between 1 mg and 100 mg of the crystalline form of a salt of OAD2 per day.
